(19) United States
(12) Patent Application Publication
(10) Pub. No.: US 2016/0040096 A1
(43) Pub. Date: Feb. 11, 2016

(54) PERFUME SYSTEMS

(71) Applicant: The Procter & Gamble Company, Cincinnati, OH (US)

(72) Inventors: Johan SMETS, Lubbeek (BE); Patricia Ann BLONDIN, Cincinnati, OH (US); Hugo Robert Germain DENUTTE, Hofstade (Aalst) (BE); Bernard EGGER, Bernex (CH); Wilhelm PICKENHAGEN, Chavannes-des-Bois (CH)

(21) Appl. No.: 14/918,630

(22) Filed: Oct. 21, 2015

Related U.S. Application Data
(63) Continuation of application No. PCT/US2014/034765, filed on Apr. 21, 2014.

(51) Int. Cl. C11B 9/00; A61Q 5/02; C11D 3/50; A61K 8/49

(52) U.S. Cl. CPC C11B 9/008; A61K 8/4973; A61K 8/498; A61Q 5/02; C11D 3/50; C11B 9/0076

(57) ABSTRACT

The present application relates to perfume raw materials, perfume delivery systems and consumer products comprising such perfume raw materials and/or such perfume delivery systems, as well as processes for making and using such perfume raw materials, perfume delivery systems and consumer products. Such perfume raw materials and compositions, including the delivery systems, disclosed herein expand the perfume communities' options as such perfume raw materials can provide variations on character and such compositions can provide desired odor profiles.

### FIELD OF INVENTION

The present application relates to perfume raw materials, perfume delivery systems and consumer products comprising such perfume raw materials and/or perfume delivery systems, as well as processes for making and using such perfume raw materials, perfume delivery systems and consumer products.

### BACKGROUND OF THE INVENTION

Consumer products may comprise one or more perfumes and/or perfume delivery systems that can mask an undesirable odor and/or provide a desired scent to a product and/or a situs that is contacted with such a product. While current perfumes and perfume delivery systems provide desirable fragrances, consumers continue to seek products that have scents that may be longer lasting and that are tailored to their individual desires (see for example USPA 2007/0275866 A1 and USPA 2008/0305977 A1)—unfortunately the pool of perfume raw materials and perfume delivery systems that is available is still too limited to completely meet the perfume community's needs. Thus, perfumers need an ever larger pool of perfume raw materials and perfume delivery systems.

Applicants believe that the perfume raw materials and perfumes, including the delivery systems, disclosed herein expand the perfume community's options, as such perfume raw materials can provide variations on character and such perfumes can provide desired odor profiles in consumer products. In certain aspects, such perfume delivery systems comprising such perfume raw materials may provide variations on character and/or odor profiles that are better than expected as measured by parameters such as headspace analysis (employed to determine perfume delivery system perfume leakage and/or perfume delivery efficiency), ClogP, boiling point and/or odor detection threshold.

### SUMMARY OF THE INVENTION

The present application relates to perfume raw materials, perfume delivery systems and consumer products comprising such perfume raw materials and/or such perfume delivery systems, as well as processes for making and using such perfume raw materials, perfume delivery systems and consumer products.

### DETAILED DESCRIPTION OF THE INVENTION

### Definitions

As used herein “consumer product” means baby care, beauty care, fabric & home care, family care, feminine care, health care, snack and/or beverage products or devices generally intended to be used or consumed in the form in which it is sold. Such products include but are not limited to diapers, bibs, wipes; products for and/or methods relating to treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use including fine fragrances; and shaving products, products for and/or methods relating to treating fabrics, hard surfaces and any other surfaces in the area of fabric and home care, including: air care including air fresheners and scent delivery systems, car care, dishwashing, fabric conditioning (including softening and/or freshening), laundry detergency, laundry and rinse additive and/or care, hard surface cleaning and/or treatment including floor and toilet bowl cleaners, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, RX pharmaceuticals, pet health and nutrition; processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, corn snacks, pellet snacks, extruded snacks and bagel chips); and coffee.

As used herein, the term “cleaning and/or treatment composition” is a subset of consumer products that includes, unless otherwise indicated, beauty care, fabric & home care products. Such products include, but are not limited to, products for treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use including fine fragrances; and shaving products, products for treating fabrics, hard surfaces and any other surfaces in the area of fabric and home care, including: air care including air fresheners and scent delivery systems, car care, dishwashing, fabric conditioning (including softening and/or freshening), laundry detergency, laundry and rinse additive and/or care, hard surface cleaning and/or treatment including floor and toilet bowl cleaners, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, dentifrice, car or carpet shampoos, bathroom cleaners including toilet bowl cleaners; hair shampoos and hair-rinses; shower gels, fine fragrances and foam baths and metal cleaners; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists all for consumer or/and institutional use; and/or methods relating to oral care including toothpastes, tooth gels, tooth rinses, denture adhesives, tooth whitening.

As used herein, the term “fabric and/or hard surface cleaning and/or treatment composition” is a subset of cleaning and treatment compositions that includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, car or carpet shampoos, bathroom cleaners including toilet bowl cleaners; and metal cleaners, fabric conditioning products including softening and/or freshening that may be in liquid, solid and/or dryer sheet form ; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists. All of such products which were applicable may be in standard, concentrated or even highly concentrated form even to the extent that such products may in certain aspect be non-aqueous.

As used herein, articles such as “a” and “an” when used in a claim, are understood to mean one or more of what is claimed or described.

As used herein, the terms “include”, “includes” and “including” are meant to be non-limiting.

As used herein, the term “solid” includes granular, powder, bar and tablet product forms.

As used herein, the term “fluid” includes liquid, gel, paste and gas product forms.

As used herein, the term “situs” includes paper products, fabrics, garments, hard surfaces, hair and skin.

As used herein, “perfume raw materials” include molecules that can serve the purposes of providing odour and/or a sensation such as cooling.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

TABLE 1
Molecules (also known as ″PRMs″)
Suitable molecules include the PRMs listed in
Table 1 below and stereoisomers thereof.

| | | | |
|---|---|---|---|
| 1 | 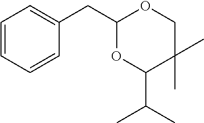 [SMILES: CC(C)C1OC(Cc2ccccc2)OCC1(C)C] | 2-benzyl, 4-isopropyl, 5-dimethyl, m-dioxane | Fruity, rose, honey |
| 2 | 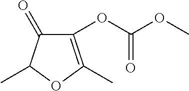 | Furaneol carbonate | Sweet, furaneol- maltol, caramel |

The PRMs disclosed in Table 1 above (a.k.a., molecules—as referred to in the Examples section) may provide one or more of the following benefits at levels that Applicants believe are unexpected in view of PRMs in general: neat product odor; wet fabric odor when applied to a fabric; dry fabric odor when applied to a fabric; reduced leakage from an encapsulate, including an encapsulate such as a perfume microcapsule; increased head space versus neat oil in certain perfume delivery technologies; odor when used in a matrix perfume delivery that is applied to a package; neat product odor when applied to a cleaning and/or treatment composition; fine fragrance composition odor when used in a fine fragrance; dry hair odor when a composition comprising such a PRM is applied to hair; PRM bloom from a solution comprising such a PRM; and new PRM character when applied to a situs. Confirmation of such benefits can be obtained by applying standard test methodologies detailed herein. The PRMs and stereoisomers of such PRMs disclosed in Table 1 above can be made in accordance with the teachings detailed in the present specification. Moreover, these PRMs are liquid at room temperature. As a result, their t application in perfumes and products is facilitated.

In one aspect, the PRMs disclosed in Table 1 and stereoisomers thereof are suitable for use, as defined by the present specification, in consumer products at levels, based on total consumer product weight, of from about 0.0001% to about 25%, from about 0.0005% to about 10%, from about 0.001% to about 5%, from about 0.005% to about 2.5%, or even from 0.01% to about 1%. Such PRMs and stereoisomers thereof may be used in various combinations in the aforementioned consumer products. In one aspect, a consumer product may comprise one or more PRMs selected from Table 1 PRMs Nos. 1 and 2 and stereoisomers thereof.

In another aspect, the PRMs disclosed in Table 1 and stereoisomers thereof are suitable for use, as defined by the present specification, in cleaning and/or treatment compositions at levels, based on total cleaning and treatment products weight of from about 0.0001% to about 25%, from about 0.0005% to about 10%, from about 0.001% to about 5%, from about 0.005% to about 2.5%, or even from 0.01% to about 1%. Such PRMs and stereoisomers thereof may be used in various combinations in the aforementioned cleaning and/ treatment compositions. In one aspect, a cleaning and/or treatment composition may comprise one or more PRMs selected from Table 1 PRMs Nos. 1 and 2 and stereoisomers thereof.

In another aspect, the PRMs disclosed in Table 1 and stereoisomers thereof are suitable for use, as defined by the present specification, in fabric and/or hard surface cleaning and/or treatment compositions at levels, based on total fabric and/or hard surface cleaning and/or treatment composition weight of from about 0.00001% to about 25%, from 0.00005% to about 10%, from 0.0001% to about 5%, from 0.0005% to about 1.0%, or even from 0.001% to about 0.5%. Such PRMs and stereoisomers thereof may be used in various combinations in the aforementioned fabric and/or hard surface cleaning and/or treatment compositions. In one aspect, a fabric and/or hard surface cleaning and/or treatment composition may comprise one or more PRMs selected from Table 1 PRMs Nos. 1 and 2 and stereoisomers thereof.

In another aspect, a detergent that may comprise the same level of the PRMs as disclosed for the aforementioned fabric and hard surface cleaning and/or treatment compositions is disclosed. In one aspect, a detergent may comprise one or more PRMs selected from Table 1 PRMs Nos. 1 and 2 and stereoisomers thereof.

In another aspect, the PRMs disclosed in Table 1 and stereoisomers thereof are suitable for use in highly compacted consumer products, including highly compacted fabric and hard surface cleaning and/or treatment compositions. For example, the PRMs disclosed in Table 1 and stereoisomers thereof may be employed in solid or fluid highly compacted detergents at levels of from about 0.00001% to about 25%, from 0.00005% to about 10%, from 0.0001% to about 5%, from 0.0005% to about 1.0%, or even from 0.001% to about 0.5%, based on total composition weight. Such PRMs and stereoisomers thereof may be used in various combinations in the aforementioned highly compacted detergent compositions. Such highly compact detergents typically comprise a higher than normal percentage of active ingredients. In one aspect, a highly compacted detergent may comprise one or more PRMs selected from Table 1 PRMs Nos. 1 and 2 and stereoisomers thereof.

### Perfume Delivery Systems

Certain perfume delivery systems, methods of making certain perfume delivery systems and the uses of such perfume delivery systems are disclosed in USPA 2007/0275866 A1. Such perfume delivery systems include:

### I. Polymer Assisted Delivery (PAD):

This perfume delivery technology uses polymeric materials to deliver perfume materials. Examples of PAD include employment of classical coacervation, water soluble or partly soluble to insoluble charged or neutral polymers, liquid crystals, hot melts, hydrogels, perfumed plastics, microcapsules, nano- and micro-latexes, polymeric film formers, and polymeric absorbents, etc. Further, PAD includes, but is not limited to:

a.) Matrix Systems: The perfume is dissolved or dispersed in a polymer matrix or particle. Perfume materials may be 1) dispersed into the polymer prior to formulating into the product or 2) added separately from the polymer during or after formulation of the product. Suitable organic latex particles include a wide range of materials including, but not limited to, polyacetal, polyacrylate, polyamide, polybutadiene, polychloroprene, polyethylene, polycyclohexylene polycarbonate, polyhydroxyalkanoate, polyketone, polyester, polyetherimide, polyethersulfone, polyethylenechlorinates, polyimide, polyisoprene, polylactic acid, polyphenylene, polyphenylene, polypropylene, polystyrene, polysulfone, polyvinyl acetate, polyvinyl chloride, as well as polymers or copolymers based on amine, acrylonitrile-butadiene, cellulose acetate, ethylene-vinyl acetate, ethylene vinyl alcohol, styrene-butadiene, vinyl acetate-ethylene, and mixtures thereof. All such matrix systems may include, for example, polysaccharides and nanolatexes that may be combined with other perfume delivery technologies, including other PAD systems such as PAD reservoir systems in the form of a perfume microcapsule (PMC). Silicone-assisted delivery (SAD) may also be used. Examples of silicones include polydimethylsiloxane and polyalkyldimethylsiloxanes. Other examples include those with amine functionality, which may be used to provide benefits associated with amine-assisted delivery (AAD) and/or polymer-assisted delivery (PAD) and/or amine-reaction products (ARP).

b.) Reservoir Systems: Reservoir systems are also known as core-shell systems (e.g., perfume microcapsules). In such a system, the benefit agent is surrounded by a benefit agent release controlling membrane, which may serve as a protective shell. Suitable shell materials include reaction products of one or more amines with one or more aldehydes, such as urea cross-linked with formaldehyde or gluteraldehyde, melamine cross-linked with formaldehyde, gelatin-polyphosphate coacervates optionally cross-linked with gluteraldehyde, gelatin-gum arabic coacervates, cross-linked silicone fluids, polyamine reacted with polyisocyanates, polyamines reacted with epoxides, polyvinyl alcohol cross linked with gluteraldehyde, polydivinyl chloride, polyesters, polyamides, polyacrylates and mixtures thereof. In one aspect, said polyacrylate based materials may comprise polyacrylate formed from methylmethacrylate/dimethylaminomethyl methacrylate, polyacrylate formed from amine acrylate and/or methacrylate and strong acid, polyacrylate formed from carboxylic acid acrylate and/or methacrylate monomer and strong base, polyacrylate formed from an amine acrylate and/or methacrylate monomer and a carboxylic acid acrylate and/or carboxylic acid methacrylate monomer, and mixtures thereof.

Suitable core materials include perfume compositions, perfume raw materials, silicone oils, waxes, hydrocarbons, higher fatty acids, essential oils, lipids, skin coolants, vitamins, sunscreens, antioxidants, glycerine, catalysts, bleach particles, silicon dioxide particles, malodor reducing agents, odor-controlling materials, chelating agents, antistatic agents, softening agents, insect and moth repelling agents, colorants, antioxidants, chelants, bodying agents, drape and form control agents, smoothness agents, wrinkle control agents, sanitization agents, disinfecting agents, germ control agents, mold control agents, mildew control agents, antiviral agents, drying agents, stain resistance agents, soil release agents, fabric refreshing agents and freshness extending agents, chlorine bleach odor control agents, dye fixatives, dye transfer inhibitors, color maintenance agents, optical brighteners, color restoration/rejuvenation agents, anti-fading agents, whiteness enhancers, anti-abrasion agents, wear resistance agents, fabric integrity agents, anti-wear agents, anti-pilling agents, defoamers and anti-foaming agents, UV protection agents for fabrics and skin, sun fade inhibitors, anti-allergenic agents, enzymes, water proofing agents, fabric comfort agents, shrinkage resistance agents, stretch resistance agents, stretch recovery agents, skin care agents, glycerin, and natural actives such as aloe vera, vitamin E, shea butter, cocoa butter, and the like, brighteners, antibacterial actives, antiperspirant actives, cationic polymers, dyes and mixtures thereof. Suitable perfume compositions may comprise enduring perfumes, such as perfume raw materials that have a cLogP greater than about 2.5 and a boiling point greater than about 250° C. Further, suitable perfume compositions may comprise blooming perfumes that comprise perfume raw materials that have a cLogP of greater than about 3 and a boiling point of less than about 260° C.

Suitable core materials can be stabilized and/or emulsified in solvent systems with organic or inorganic materials (organic materials can be polymers of anionic nature, non-ionic nature or cationic nature, like polyacrylates and polyvinyl alcohol). Suitable processes to make core-shell systems include coating, extrusion, spray drying, interfacial polymerization, polycondensation, simple coacervation, complex coacervation, free radical polymerization, in situ emulsion polymerization, matrix polymerization and combinations thereof.

Suitable characteristics for the core-shell systems include:
a) a shell thickness of from about 20 nm to about 500 nm, from about 40 nm to about 250 nm, or from about 60 nm to about 150 nm;
b) a shell core ratio of from about 5:95 to about 50:50, from about 10:90 to about 30:70, or from about 10:90 to about 15:85;
c) a fracture strength of from about 0.1 MPa to about 16 MPa, from about 0.5 MPa to about 8 MPa, or even from about 1 MPa to about 3 MPa; and
d) an average particle size of from about 1 micron to about 100 microns, from about 5 microns to about 80 microns, or even from about 15 microns to about 50 microns.

Suitable deposition and/or retention enhancing coatings that may be applied to the core-shell systems include non-ionic polymers, anionic polymers, cationic polymers such as polysaccharides including, but not limited to, cationically modified starch, cationically modified guar, polysiloxanes, poly diallyl dimethyl ammonium halides, copolymers of poly diallyl dimethyl ammonium chloride and vinyl pyrrolidone, acrylamides, imidazoles, imidazolinium halides, imidazolium halides, poly vinyl amine, copolymers of poly vinyl amine and N-vinyl formamide and mixtures thereof. In another aspect, suitable coatings may be selected from the group consisting of polyvinylformaldehyde, partially hydroxylated polyvinylformaldehyde, polyvinylamine, polyethyleneimine, ethoxylated polyethyleneimine, polyvinylalcohol, polyacrylates and combinations thereof.

Suitable methods of physically reducing and/or removing any residual type materials from the core-shell making process may be employed, such as centrifugation. Suitable methods of chemically reducing any residual type materials may also be employed, such as the employment of scavengers, for example formaldehyde scavengers including sodium bisulfite, urea, ethylene urea, cysteine, cysteamine, lysine, glycine, serine, carnosine, histidine, glutathione, 3,4-diaminobenzoic acid, allantoin, glycouril, anthranilic acid, methyl anthranilate, methyl 4-aminobenzoate, ethyl acetoacetate, acetoacetamide, malonamide, ascorbic acid, 1,3-dihydroxyacetone dimer, biuret, oxamide, benzoguanamine, pyroglutamic acid, pyrogallol, methyl gallate, ethyl gallate, propyl gallate, triethanol amine, succinamide, thiabendazole, benzotriazol, triazole, indoline, sulfanilic acid, oxamide, sorbitol, glucose, cellulose, poly(vinyl alcohol), partially hydrolyzed poly(vinylformamide), poly(vinyl amine), poly(ethylene imine), poly(oxyalkyleneamine), poly(vinyl alcohol)-co-poly(vinyl amine), poly(4-aminostyrene), poly(l-lysine), chitosan, hexane diol, ethylenediamine-N,N′-bisacetoacetamide, N-(2-ethylhexyl)acetoacetamide, 2-benzoylacetoacetamide, N-(3-phenylpropyl)acetoacetamide, lilial, helional, melonal, triplal, 5,5-dimethyl-1,3-cyclohexanedione, 2,4-dimethyl-3-cyclohexenecarboxaldehyde, 2,2-dimethyl-1,3-dioxan-4,6-dione, 2-pentanone, dibutyl amine, triethylenetetramine, ammonium hydroxide, benzylamine, hydroxycitronellol, cyclohexanone, 2-butanone, pentane dione, dehydroacetic acid, or mixtures thereof.

II. Molecule-Assisted Delivery (MAD): Non-polymer materials or molecules may also serve to improve the delivery of perfume. Without wishing to be bound by theory, perfume may non-covalently interact with organic materials, resulting in altered deposition and/or release. Non-limiting examples of such organic materials include but are not limited to hydrophobic materials such as organic oils, waxes, mineral oils, petrolatum, fatty acids or esters, sugars, surfactants, liposomes and even other perfume raw material (perfume oils), as well as natural oils, including body and/or other soils.

III. Fiber-Assisted Delivery (FAD):

The choice or use of a situs itself may serve to improve the delivery of perfume. In fact, the situs itself may be a perfume delivery technology. For example, different fabric types such as cotton or polyester will have different properties with respect to ability to attract and/or retain and/or release perfume. The amount of perfume deposited on or in fibers may be altered by the choice of fiber, and also by the history or treatment of the fiber, as well as by any fiber coatings or treatments. Fibers may be pre-loaded with a perfume, and then added to a product that may or may not contain free perfume and/or one or more perfume delivery technologies.

IV. Amine Assisted Delivery (AAD): The amine-assisted delivery technology approach utilizes materials that contain an amine group to increase perfume deposition or modify perfume release during product use. There is no requirement in this approach to pre-complex or pre-react the perfume raw material(s) and the amine prior to addition to the product. In one aspect, amine-containing AAD materials suitable for use herein may be non-aromatic, for example, polyalkylimine, such as polyethyleneimine (PEI), or polyvinylamine (PVAm); or aromatic, for example, anthranilates. Such materials may also be polymeric or non-polymeric. In one aspect, such materials contain at least one primary amine. In another aspect, a material that contains a heteroatom other than nitrogen, for example sulfur, phosphorus or selenium, may be used as an alternative to amine compounds. In yet another aspect, the aforementioned alternative compounds can be used in combination with amine compounds. In yet another aspect, a single molecule may comprise an amine moiety and one or more of the alternative heteroatom moieties, for example, thiols, phosphines and selenols.

V. Cyclodextrin Delivery System (CD): This technology approach uses a cyclic oligosaccharide or cyclodextrin to improve the delivery of perfume. Typically a perfume and cyclodextrin (CD) complex is formed. Such complexes may be preformed, formed in-situ, or formed on or in the situs.

VI. Starch Encapsulated Accord (SEA): SEA's are starch encapsulated perfume materials. Suitable starches include modified starches such as hydrolyzed starch, acid thinned starch, starch having hydrophobic groups, such as starch esters of long chain hydrocarbons (C₅ or greater), starch acetates, starch octenyl succinate and mixtures thereof. In one aspect, starch esters, such as starch octenyl succinates, are employed. Suitable perfumes for encapsulation include the HIA perfumes, including those having a boiling point determined at the normal standard pressure of about 760 mmHg of 275° C. or lower, an octanol/water partition coefficient P of about 2000 or higher and an odor detection threshold of less than or equal 50 parts per billion (ppb). In one aspect, the perfume may have logP of 2 or higher.

VII. Inorganic Carrier Delivery System (ZIC): This technology relates to the use of porous zeolites or other inorganic materials to deliver perfumes. Perfume-loaded zeolite may be used with or without adjunct ingredients used for example to coat the perfume-loaded zeolite (PLZ) to change its perfume release properties during product storage or during use or from the dry situs. Suitable zeolite and inorganic carriers as well as methods of making same may be found in USPA 2005/0003980 A1. Silica is another form of ZIC. Another example of a suitable inorganic carrier includes inorganic tubules, where the perfume or other active material is contained within the lumen of the nano- or micro-tubules. In one aspect, the perfume-loaded inorganic tubule (or Perfume-Loaded Tubule or PLT) is a mineral nano- or micro-tubule, such as halloysite or mixtures of halloysite with other inorganic materials, including other clays. The PLT technology may also comprise additional ingredients on the inside and/or outside of the tubule for the purpose of improving in-product diffusion stability, deposition on the desired situs or for controlling the release rate of the loaded perfume. Monomeric and/or polymeric materials, including starch encapsulation, may be used to coat, plug, cap, or otherwise encapsulate the PLT. Suitable PLT systems as well as methods of making same may be found in U.S. Pat. No. 5,651,976.

VIII. Pro-Perfume (PP): This technology refers to perfume technologies that result from the reaction of perfume materials with other substrates or chemicals to form materials that have a covalent bond between one or more PRMs and one or more carriers. The PRM is converted into a new material called a pro-PRM (i.e., pro-perfume), which then may release the original PRM upon exposure to a trigger such as water or light. Non-limiting examples of pro-perfumes include Michael adducts (e.g., beta-amino ketones), aromatic or non-aromatic imines (Schiffs Bases), oxazolidines, beta-keto esters, and orthoesters. Another aspect includes compounds comprising one or more beta-oxy or beta-thio carbonyl moieties capable of releasing a PRM, for example, an alpha, beta-unsaturated ketone, aldehyde or carboxylic ester.

a.) Amine Reaction Product (ARP): For purposes of the present application, ARP is a subclass or species of PP. One may also use “reactive” polymeric amines in which the amine functionality is pre-reacted with one or more PRMs, typically PRMs that contain a ketone moiety and/or an aldehyde moiety, to form the ARP. Typically, the reactive amines are primary and/or secondary amines, and may be part of a polymer or a monomer (non-polymer). Such ARPs may also be mixed with additional PRMs to provide benefits of polymer-assisted delivery and/or amine-assisted delivery. Non-limiting examples of polymeric amines include polymers based on polyalkylimines, such as polyethyleneimine (PEI), or polyvinylamine (PVAm). Non-limiting examples of monomeric (non-polymeric) amines include hydroxyl amines, such as 2-aminoethanol and its alkyl substituted derivatives, and aromatic amines such as anthranilates. The ARPs may be premixed with perfume or added separately in leave-on or rinse-off applications. In another aspect, a material that contains a heteroatom other than nitrogen, for example oxygen, sulfur, phosphorus or selenium, may be used as an alternative to amine compounds. In yet another aspect, the aforementioned alternative compounds can be used in combination with amine compounds. In yet another aspect, a single molecule may comprise an amine moiety and one or more of the alternative heteroatom moieties, for example, thiols, phosphines and selenols.

In one aspect, the PRMs disclosed in Table 1 and stereoisomers thereof are suitable for use in perfume delivery systems at levels, based on total perfume delivery system weight, of from 0.001% to about 50%, from 0.005% to 30%, from 0.01% to about 10%, from 0.025% to about 5%, or even from 0.025% to about 1%.

In another aspect, the perfume delivery systems disclosed herein are suitable for use in consumer products, cleaning and treatment compositions, fabric and hard surface cleaning and/or treatment compositions, detergents, and highly compacted consumer products, including highly compacted fabric and hard surface cleaning and/or treatment compositions (e.g., solid or fluid highly compacted detergents) at levels, based on total consumer product weight, from about 0.001% to about 20%, from about 0.01% to about 10%, from about 0.05% to about 5%, from about 0.1% to about 0.5%.

In another aspect, the amount of PRMs from Table 1 present in the perfume delivery systems, based on the total microcapsule and/or nanocapsule (Polymer Assisted Delivery (PAD) Reservoir System) weight, may be from about 0.1% to about 99%, from 25% to about 95%, from 30 to about 90%, from 45% to about 90%, or from 65% to about 90%. In one aspect, microcapsules and/or nanocapsules may comprise one or more PRMs selected from Table 1 PRMs Nos. 1 and 2; stereoisomers of Table 1 PRMs Nos. 1 and 2 and mixtures thereof.

In one aspect, the amount of total perfume based on total weight of starch encapsulates and starch agglomerates (Starch Encapsulated Accord (SEA)) ranges from 0.1% to about 99%, from 25% to about 95%, from 30 to about 90%, from 45% to about 90%, from 65% to about 90%. In one aspect, the PRMs disclosed in Table 1 and stereoisomers thereof are suitable for use in such starch encapsulates and starch agglomerates. Such PRMs and stereoisomers thereof may be used in combination in such starch encapsulates and starch agglomerates.

In another aspect, the amount of total perfume based on total weight of [cyclodextrin-perfume] complexes (Cyclodextrin (CD)) ranges from 0.1% to about 99%, from 2.5% to about 75%, from 5% to about 60%, from 5% to about 50%, from 5% to about 25%. In one aspect, the PRMs disclosed in Table 1 and stereoisomers thereof are suitable for use in such [cyclodextrin-perfume] complexes. Such PRMs and stereoisomers thereof may be used in combination in such [cyclodextrin—perfume] complexes.

In another aspect, the amount of total perfume based on total weight of Polymer Assisted Delivery (PAD) Matrix Systems (including Silicones) ranges from 0.1% to about 99%, from 2.5% to about 75%, from 5% to about 60%, from 5% to about 50%, from 5% to about 25%. In one aspect, the amount of total perfume based on total weight of a hot melt perfume delivery system/perfume loaded plastic Matrix System and ranges from 1% to about 99%, from 2.5% to about 75%, from 5% to about 60%, from 5% to about 50%, from 10% to about 50%. In one aspect, the PRMs disclosed in Table 1 and stereoisomers thereof are suitable for use in such Polymer Assisted Delivery (PAD) Matrix Systems, including hot melt perfume delivery system/perfume loaded plastic Matrix Systems. Such PRMs and stereoisomers thereof may be used in various combinations in such Polymer Assisted Delivery (PAD) Matrix Systems (including hot melt perfume delivery system/perfume loaded plastic Matrix Systems).

In one aspect, the amount of total perfume based on total weight of Amine Assisted Delivery (AAD) (including Aminosilicones) ranges from 1% to about 99%, from 2.5% to about 75%, from 5% to about 60%, from 5% to about 50%, from 5% to about 25%. In one aspect, the PRMs disclosed in Table 1 and stereoisomers thereof are suitable for use in such Amine Assisted Delivery (AAD) systems. Such PRMs and stereoisomers thereof may be used in various combinations in such Amine Assisted Delivery (AAD) systems. In one aspect, an Amine Assisted Delivery (AAD) system may comprise one or more PRMs, and stereoisomers thereof, selected from Table 1 PRMs Nos. 1 and 2 and mixtures thereof.

In one aspect, the amount of total perfume based on total weight of Amine Reaction Product (ARP) ranges from 1% to about 99%, from 2.5% to about 75%, from 5% to about 60%, from 5% to about 50%, from 5% to about 25%. In one aspect, the PRMs disclosed in Table 1 and stereoisomers thereof are suitable for use in such Amine Reaction Product (ARP) systems. Such PRMs and stereoisomers thereof may be used in various combinations in such Amine Reaction Product (ARP) systems. In one aspect, an Amine Reaction Product (ARP) system may comprise one or more PRMs, and stereoisomers thereof, selected from Table 1 PRM No. 2 and mixtures thereof. In another aspect, an Amine Reaction Product (ARP) system may comprise one or more PRMs, and stereoisomers thereof, selected from Table 1 PRM No. 2 and mixtures thereof.

The perfume delivery technologies (a.k.a., perfume delivery systems) that are disclosed in the present specification may be used in any combination in any type of consumer product, cleaning and/or treatment composition, fabric and hard surface cleaning and/or treatment composition, detergent, and/or highly compact detergent.

### Perfumes

The PRMs disclosed in Table 1 may be used to formulate perfumes. Such perfumes are combinations of PRMs that may comprise a combination of Table 1 PRMs, or one or more Table 1 PRMs and one or more additional PRMs. When used in a perfume, the Table 1 PRMs may be employed, based on total perfume weight, at levels of from about 0.01% to about 50%, from about 0.1% to about 15%, from about 0.1% to about 10% or even from about 0.5% to about 10%. Such perfumes may be utilized in various applications, including being applied neat to a situs or used in a consumer product, cleaning and/or treatment composition, fabric and hard surface cleaning and/or treatment composition, detergent, and/or a highly compact detergent.

### Adjunct Materials

For the purposes of the present invention, the non-limiting list of adjuncts illustrated hereinafter are suitable for use in the compositions detailed herein (e.g., consumer products, cleaning and/or treatment compositions, fabric and hard surface cleaning and/or treatment compositions, detergents, and/or a highly compact detergents). Such adjunct materials may be desirably incorporated in certain embodiments of the compositions, for example to assist or enhance performance of the composition, for treatment of the substrate to be cleaned, or to modify the aesthetics of the composition as is the case with perfumes, colorants, dyes or the like. It is understood that such adjuncts are in addition to the components that are supplied via Applicants' perfumes and/or perfume systems detailed herein. The precise nature of these additional components, and levels of incorporation thereof, will depend on the physical form of the composition and the nature of the operation for which it is to be used.

Suitable adjunct materials include, but are not limited to, surfactants, builders, chelating agents, dye transfer inhibiting agents, dispersants, enzymes, and enzyme stabilizers, catalytic materials, bleach activators, polymeric dispersing agents, clay soil removal/anti-redeposition agents, brighteners, suds suppressors, dyes, additional perfume and perfume delivery systems, structure elasticizing agents, fabric softeners, carriers, hydrotropes, processing aids and/or pigments, metal salts, structurants or binders, anti-tartar agents, anti-caries agents, abrasives, fillers, humectants, breath agents, flavors, antibacterial agents. In addition to the disclosure below, suitable examples of such other adjuncts and levels of use are found in U.S. Pat. Nos. 5,576,282, 6,306,812 B1, and 6,326,348 B1.

Each adjunct ingredient is not essential to Applicants' compositions. Thus, certain embodiments of Applicants' compositions may not contain one or more of the following adjuncts materials: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, additional perfumes and perfume delivery systems, structure elasticizing agents, fabric softeners, carriers, hydrotropes, processing aids and/or pigments, metal salts, structurants or binders, anti-tartar agents, anti-caries agents, abrasives, fillers, humectants, breath agents, flavors, antibacterial agents. However, when one or more adjuncts are present, such adjuncts may be present as detailed below:

Surfactants—The compositions according to the present invention can comprise a surfactant or surfactant system wherein the surfactant can be selected from nonionic and/or anionic and/or cationic surfactants and/or ampholytic and/or zwitterionic and/or semi-polar nonionic surfactants. The surfactant is typically present at a level of from about 0.1%, from about 1%, or even from about 5% by weight of the cleaning compositions to about 99.9%, to about 80%, to about 35%, or even to about 30% by weight of the cleaning compositions.

Builders—The compositions of the present invention can comprise one or more detergent builders or builder systems. When present, the compositions will typically comprise at least about 1% builder, or from about 5% or 10% to about 80%, 50%, or even 30% by weight, of said builder. Builders include, but are not limited to, the alkali metal, ammonium and alkanolammonium salts of polyphosphates, alkali metal silicates, alkaline earth and alkali metal carbonates, aluminosilicate builders polycarboxylate compounds. ether hydroxypolycarboxylates, copolymers of maleic anhydride with ethylene or vinyl methyl ether, 1,3,5-trihydroxybenzene-2,4,6-trisulphonic acid, and carboxymethyl-oxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, oxydisuccinic acid, polymaleic acid, benzene 1,3,5-tricarboxylic acid, carboxymethyloxysuccinic acid, and soluble salts thereof.

Chelating Agents—The compositions herein may also optionally contain one or more copper, iron and/or manganese chelating agents. If utilized, chelating agents will generally comprise from about 0.1% by weight of the compositions herein to about 15%, or even from about 3.0% to about 15% by weight of the compositions herein.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in the compositions herein, the dye transfer inhibiting agents are present at levels from about 0.0001%, from about 0.01%, from about 0.05% by weight of the cleaning compositions to about 10%, about 2%, or even about 1% by weight of the cleaning compositions.

Dispersants—The compositions of the present invention can also contain dispersants. Suitable water-soluble organic materials are the homo- or co-polymeric acids or their salts, in which the polycarboxylic acid may comprise at least two carboxyl radicals separated from each other by not more than two carbon atoms.

Enzymes—The compositions can comprise one or more detergent enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, B-glucanases, arabinosidases, hyaluronidase, chondroitinase, laccase, and amylases, or mixtures thereof. A typical combination is a cocktail of conventional applicable enzymes like protease, lipase, cutinase and/or cellulase in conjunction with amylase.

Enzyme Stabilizers—Enzymes for use in compositions, for example, detergents can be stabilized by various techniques. The enzymes employed herein can be stabilized by the presence of water-soluble sources of calcium and/or magnesium ions in the finished compositions that provide such ions to the enzymes.

Catalytic Metal Complexes—Applicants' compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra (methyl-enephosphonic acid) and water-soluble salts thereof. Such catalysts are disclosed in U.S. Pat. No. 4,430,243.

If desired, the compositions herein can be catalyzed by means of a manganese compound. Such compounds and levels of use are well known in the art and include, for example, the manganese-based catalysts disclosed in U.S. Pat. No. 5,576,282. Cobalt bleach catalysts useful herein are known, and are described, for example, in U.S. Pat. No. 5,597,936.

Compositions herein may also suitably include a transition metal complex of a macropolycyclic rigid ligand—abbreviated as “MRL”. As a practical matter, and not by way of limitation, the compositions and cleaning processes herein can be adjusted to provide on the order of at least one part per hundred million of the benefit agent MRL species in the aqueous washing medium, and may provide from about 0.005 ppm to about 25 ppm, from about 0.05 ppm to about 10 ppm, or even from about 0.1 ppm to about 5 ppm, of the MRL in the wash liquor. Suitable transition-metals in the instant transition-metal bleach catalyst include manganese, iron and chromium. Suitable MRL's herein are a special type of ultra-rigid ligand that is cross-bridged such as 5,12-diethyl-1,5,8,12-tetraazabicyclo[6.6.2]hexa-decane. Suitable transition metal MRLs are readily prepared by known procedures, such as taught for example in and U.S. Pat. No. 6,225,464.

### Methods of Use

Some of the consumer products disclosed herein can be used to clean and/or treat a situs inter alia a surface or fabric. Typically at least a portion of the situs is contacted with an embodiment of Applicants' composition, in neat form or diluted in a liquor, for example, a wash liquor and then the situs may be optionally washed and/or rinsed. In one aspect, a situs is optionally washed and/or rinsed, contacted with a composition according to the present invention and then optionally washed and/or rinsed. In one aspect, the situs is dried in a dryer, and/or passivel dried, for example, by line drying after the aforementioned cleaning and/or treating. For purposes of the present invention, washing includes but is not limited to, scrubbing, and mechanical agitation. The fabric may comprise most any fabric capable of being laundered or treated in normal consumer use conditions. Liquors that may comprise the disclosed compositions may have a pH of from about 3 to about 11.5. Such compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric ratio is typically from about 1:1 to about 30:1.

### Test Methods

It is understood that the test methods that are disclosed in the Test Methods Section of the present application should be used to determine the respective values of the parameters of Applicants' invention as such invention is described and claimed herein.

### (1) ClogP

The logP values of many perfume ingredients have been reported; for example, the Pomona92 database, available from Daylight Chemical Information Systems, Inc. (Daylight CIS), Irvine, Calif., contains many, along with citations to the original literature. However, the logP values are most conveniently calculated by the “CLOGP” program, also available from Daylight CIS. This program also lists experimental logP values when they are available in the Pomona92 database. The “calculated logP” (ClogP) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990, incorporated herein by reference). The fragment approach is based on the chemical structure of each perfume ingredient, and takes into account the numbers and types of atoms, the atom connectivity, and chemical bonding. The ClogP values, which are the most reliable and widely used estimates for this physicochemical property, are preferably used instead of the experimental logP values in the selection of perfume ingredients which are useful in the present invention.

### (2) Boiling Point

Boiling point is measured by ASTM method D2887-04a, “Standard Test Method for Boiling Range Distribution of Petroleum Fractions by Gas Chromatography,” ASTM International.

### (3) Headspace Ratio

(a) Obtain a fragrance free consumer product formulation.
(b) Obtain fragrance microcapsules whose water content has been adjusted to achieve a perfume content of 25 wt % in the aqueous slurry.
(c) Prepare Sample A by adding 2.0 grams of the fragrance microcapsule aqueous slurry to 95 grams of the fragrance free consumer product formulation. Then add 3.0 grams of deionized water to balance the formulation to 100 grams. Age this formulation for 1 week at 40 degrees Centigrade.
(d) Prepare Sample B by adding 0.50 grams of the neat fragrance to 95 grams of fragrance free consumer product formulation. Then add 4.5 grams of deionized water to balance the formulation to 100 grams. Age this formulation for 1 week at 40 degrees Centigrade.

The Headspace Ratio for determining perfume leakage from a perfume delivery system is defined as the headspace concentration of Sample A divided by the headspace concentration of

### Sample B,

$$\frac{{H}_{Sample\_A}}{{H}_{Sample\_B}} ,$$

where Hₛₐₘₚₗₑ_{_{—}}_{A} is the headspace concentration of a consumer product formulation Sample A, and Hₛₐₘₚₗₑ_{_{—}}_{B} is the headspace concentration of a consumer product formulation Sample B.

The Headspace Ratio for determining perfume delivery efficiency from a perfume delivery system is defined as the headspace concentration of Sample B divided by the headspace concentration of Sample A,

$$\frac{{H}_{Sample\_B}}{{H}_{Sample\_A}} ,$$

where H_{Sample}_{—A} is the headspace concentration of a consumer product formulation Sample A, and H_{Sample}_{_{—}}_{B} is the headspace concentration of a consumer product formulation Sample B.

Solid-Phase Micro-Extraction (SPME)-Gas Chromatography/Mass Spectrometry is used to measure the level of perfume raw materials in the headspace of products. 1.0 grams of the 1 week at 40 degrees Centigrade aged sample are placed into a clean 20 ml headspace vial and allowed to equilibrate for at least 2 hours at room temperature.

The samples are then analyzed using the MPS2-SMPE-GC-MS analysis system (GC-02001-0153, MSD-02001-0154, MPS2-02001-0155).

### Apparatus:

1. 20 ml headspace vial
2. Timer.
3. Gas Chromatograph (GC): Agilent model 6890 with a CIS-4 injector (Gerstel, Mulheim, Germany) and MPS-2 Autosampler and TDU. For SPME analysis, we used the split/splitless injector (not the CIS-4 injector).
4. GC column: J&W DB-5 MS, 30 M×0.25 mm ID, 1.0 m film thickness obtained from J&W Scientific of Folsom, Calif., USA.
5. Carrier gas, helium, 1.5 ml/min. flow rate.
6. The injector liner is a special SPME liner (0.75 mm ID) from Supelco.
7. The Detector is a model 5973 Mass Selective Detector obtained from Agilent Technologies, Inc., Wilmington, Del., USA having a source temperature of about 230° C., and a MS Quad temperature of about 150° C.
   Analysis procedure:
1. Transfer sample to proper sample tray and proceed with SPME-GC-MS analysis.
2. Start sequence of sample loading and analysis. In this step, the sample is allowed to equilibrate for at least two hours on the auto sampler tray, then sampled directly from the tray. The SPME fiber assembly is DVB/CAR/PDMS (50/30 um, 24 ga, 1 cm length). Sampling time is 5 minutes.
3. Injector temperature is at 260 C.
4. Then GC-MS analysis run is started. Desorption time is 5 minutes.
5. The following temperature program is used:
   i) an initial temperature of about 50° C. which is held for 3 minutes,
   ii) increase the initial temperature at a rate of about 6° C./min until a temperature of about 250° C. is reached, then 25° C./min to 275° C., hold at about 275° C. for 4.67 minute.
6. Perfume compounds are identified using the MS spectral libraries of John Wiley & Sons and the National Institute of Standards and Technology (NIST), purchased and licensed through Hewlett Packard.
7. Chromatographic peaks for specific ions are integrated using the Chemstation software obtained from Agilent Technologies, Inc., Wilmington, Del., USA.
8. The ratio for each PRM is calculated by dividing the peak area for the perfume raw material in Sample A by the peak area in Sample B.
9. Each ratio is then weighted by that perfume raw material's weight composition in the perfume.
10. The Headspace Ratio is calculated as the sum of the individual perfume raw material ratios obtained in step 9.
   (4) Perfume leakage can also be evaluated via% liquid-liquid extraction and gas chromatographic-mass spectrometric analysis

When determining the % perfume leakage from Perfume Microcapsules in liquid detergent, a fresh sample of liquid detergent with equal level of free perfume (without Perfume Microcapsules) must also be analyzed in parallel for reference.

1. Preparation of an internal standard solution
Stock solution of tonalid: Weigh 70 mg tonalid and add 20 ml hexane p.a.
Internal Standard Solution solution: Dilute 200 μl of stock solution in 20 ml hexane p.a.
Mix to homogenize

2. Perfume extraction from liquid detergent without perfume microcapsules (reference)
Weigh 2 g of liquid detergent product into an extraction vessel
Add 2 ml of Internal Standard Solution and close vessel
Extract perfume by gently turning the extraction vessel upside-down for 20 times (manually)
Add spoon tip of Sodium Sulphate
After separation of layers, immediately transfer hexane-layer into Gas Chromatograph auto sampler-vial and cap vial
Inject splitless (1.5 μl) into Gas Chromatograph injection-port
Run Gas Chromatographic-Mass Spectrometric analysis

3. Perfume extraction from liquid detergent with perfume microcapsules
Weigh 2 g of liquid detergent product into an extraction vessel
Add 2 ml of Internal Standard Solution and close vessel
Extract perfume by gently turning the extraction vessel upside-down for 20 times (manually)
Add spoon tip of Sodium Sulphate
After separation of layers, immediately transfer hexane-layer into Gas Chromatograph auto sampler-vial and cap vial
Inject splitless (1.5 μl) into Gas Chromatograph injection-port
Run Gas Chromatographic-Mass Spectrometric analysis

4. Calculation
The perfume leakage from capsules per individual Perfume Raw Material:

% perfume leakage=((Area Perfume Raw Material caps×Area Internal Standard Solution ref×Weight ref)/(Area Internal Standard Solution caps×Area Perfume Raw Material ref×Weight caps))×100

### (5) Odor Detection Threshold (ODT)

Determined using a gas chromatograph. The gas chromatograph is calibrated to determine the exact volume of material injected by the syringe, the precise split ratio, and the hydrocarbon response using a hydrocarbon standard of known concentration and chain length distribution. The air flow rate is accurately measured and, assuming the duration of human inhalation to last 12 seconds, the sampled volume is calculated. Since the precise concentration at the detector at any point in time is known, the mass per volume inhaled is known, and hence the concentration of material.

For example, to determine whether a material has a threshold below 50 parts per billion, solutions are delivered to the sniff port at the calculated concentration. A panelist sniffs the GC effluent and identifies the retention time when odor is noticed. The average among 6 panelists determines the threshold of notice ability. The necessary amount of analyte is injected into the column to achieve a 50 parts per billion concentration at the detector. Typical gas chromatograph parameters for determining odor detection thresholds are listed below:

GC: 5890 Series II with FID detector, 7673 Autosampler

Column: J&W Scientific DB-1

Length: 30 meters, 0.25millmeter inside diameter, 1 micrometer film thickness

Method:
split injection: 17/1 split ratio
Autosampler: 1.13 microliters per injection
Column flow: 1.10 milliLiters per minute
Air Flow: 345 milliLiters per minute
Inlet Temperature: 245 degrees Centigrade
Detector Temperature: 285 degrees Centigrade
Initial Temperature=50 degrees Centigrade, 5 degrees Centigrade per minute ramp rate, final temperature=280 degrees Centigrade, Final time=6 minutes
Leading assumptions: 12 seconds per sniff, GC air adds to sample dilution

### EXAMPLES

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### Example 1

### Synthesis of Table 1 Molecules

Synthesis of Table 1 molecule 1:

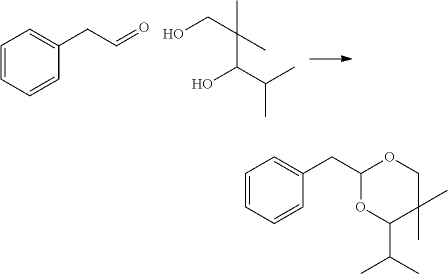

A mixture containing 30 g of 2-phenylacetaldehyde, 36.5 g of 2,2,4-trimethylpentane-1,3-diol, 0.3 g of 4-Toluenesulfonic acid monohydrate and 230 ml of toluene were put in a 500 mL three necks round flask. The mixture was heated at reflux for 5 h with continuous azeotropic removal of water by a Dean-Stark. Then, the reaction was cooled and the organic layer was washed with saturated aqueous sodium bicarbonate, then saturated brine and dried over anhydrous magnesium sulfate, filtered and concentrated to give 58 g of crude material. The purification was done by distillation on a Vigreux column (120° C./0.4mbar) to give 43 g of the desired compound as colorless oil.

¹³C NMR: 19.0 (q); 19.4 (q); 22.4 (q); 22.5 (q); 29.0 (d); 33.5 (s); 41.6 (t); 79.4 (t); 89.3 (d); 102.7 (d); 126.2 (d); 128.0 (d); 129.8 (d); 137.0 (s).

¹H-NMR: 0.72 (s, 3H); 0.92 (d, J=1.6, 3H); 0.94 (d, J=1.6, 3H); 1.08 (s, 3H); 1.83 (in, 1H); 2.87-3.00 (in, 3 H); 3.29 (d, J=11.3, 1H); 3.45 (d, J=11.1, 1H); 4.61 (dd, J=5.5, 4.6, 1H); 7.20 (in, 1H); 7.26 (in, 4H).
MS: 248 (0.1), 247 (0.6), 193 (2), 157 (45), 121 (19), 120 (41), 111 (48), 103 (6), 92 (59), 91 (100), 85 (14), 83 (10), 73 (11), 69 (25), 65 (9), 57 (19), 56 (20), 55 (18), 43 (11).
   Synthesis of Table 1 molecule 2:

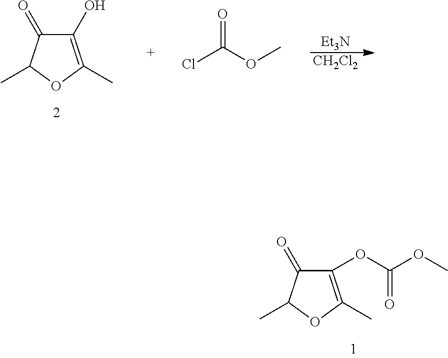

A clean, dry 1.5 L three-neck reaction flask fitted with a condenser, mechanical agitator and thermometer was purged with argon. To it were charged dichloromethane (360 mL) and 4-hydroxy-2,5-dimethylfuran-3(2H)-one (2) (60.0 g, 97%, 454 mmol, 1.0 eq). The agitator was turned on, the reaction mixture was blanketed with argon and the flask was cooled to an internal temperature of −17° C. (using a mixture of ice and dry-ice). To this clear pale orange solution was added, by means of a syringe, over 10 minutes triethylamine (55.7 g, 99%, 545 mmol, 1.2 eq). The reaction mixture changed to a yellow colour. The internal temperature was reduced to −24° C. and to the clear yellow solution was added methyl chloroformate (41.2 g, 99%, 432 mmol, 0.95 eq) over two hours via a syringe using a syringe pump. During the addition the internal temperature rose from −24° C. to −17° C. The reaction mixture changed progressively from a clear yellow solution to a cloudy yellow suspension. The reaction mixture was stirred for 2 h30 while allowing the internal temperature to rise from −17° C. to +7° C. The reaction mixture was diluted with ethyl acetate (1.5 L). The organic phase was then washed with water (7×1 L) till a GC analysis of the organic phase indicated the complete absence of 2. The organic phase was concentrated under reduced pressure (45° C., 100 mbar 3 mbar) to obtain the crude 1 as a viscous yellow oil (55.76 g, 98.0% pure by GC). The crude was flash distilled (111-152° C. pot, 93-95° C. vapour, 0.6 mbar) to obtain 1 as a very pale yellow viscous liquid (55.3 g, 98.5% pure by GC; 63% yield). The semi-pure 1 obtained from several batches run as detailed above was combined (477.7 g) and purified by fractional distillation through a 7 cm packed (glass beads) column (110-121° C. pot, 92-95° C. vapour, 0.6-0.7 mbar). All fractions were analysed by GC and those that had a purity in excess of 98.5% were combined to obtain the pure 1 as a very pale yellow oil (466.8 g, 98.8% pure).

1H-NMR (400 MHz, CDCl₃, δ): 1.49 (d, J=7.2 Hz, 3H), 2.23 (d, J=0.8 Hz, 3H), 3.89 (s, 3H), 4.59 (dq, J=0.8, 7.2 Hz, 1H)

13C-NMR (100 MHz, CDCl₃, δ): 13.8 (q), 16.3 (q), 56.0 (q), 81.5 (d), 130.0 (s), 152.9 (s), 180.1 (s), 195.1 (s).

MS (EI, 70 eV): 187 (4.5), 186 (41.4), 143 (7.5), 142 (39.8), 141 (11.3), 127 (10.7), 125 (6.6), 114 (7.1), 113 (3.6), 112 (5.9), 100 (2.8), 99 (42.6), 97 (3.2), 85 (8.3), 84 (6.2), 83 (7.6), 81 (3.0), 72 (6.7), 71 (100), 70 (11.0), 69 (22.5), 68 (4.4), 67 (5.3), 59 (40.0), 57 (4.8), 56 (23.1), 55 (19.6), 54 (5.2), 53 (5.9).

### Example 2

### Preformed Amine Reaction Product

The following ingredients are weighted off in a glass vial:
1. 50% of the perfume material comprising one or more Table 1 PRMs
2. 50% of Lupasol WF (CAS#09002-98-6) from BASF, is put at 60° C. in warm water bath for 1 hour before use.

Mixing of the two ingredients is done by using the Ultra-Turrax T25 Basic equipment (from IKA) during 5 minutes. When the mixing is finished the sample is put in a warm water bath at 60° C. for ±12 hours. A homogenous, viscous material is obtained.

In the same way as described above different ratios between the components can be used:

Weight %

| | | | | | |
|---|---|---|---|---|---|
| Perfume Material | 40 | 50 | 60 | 70 | 80 |
| Lupasol WF | 60 | 50 | 40 | 30 | 20 |

### Example 3

### 84wt % Core/16 wt % Wall Melamine Formaldehyde (MF) Capsule (PAD Reservoir System

17 grams of butyl acrylate-acrylic acid copolymer emulsifier (Colloid C351, 25% solids, pka 4.5-4.7, (Kemira Chemicals, Inc. Kennesaw, Ga. U.S.A.) and 17 grams of polyacrylic acid (35% solids, pKa 1.5-2.5, Aldrich) are dissolved and mixed in 200 grams deionized water. The pH of the solution is adjusted to pH of 6.0with sodium hydroxide solution. 7grams of partially methylated methylol melamine resin (Cymel 385, 80% solids, (Cytec Industries West Paterson, N.J., U.S.A.)) is added to the emulsifier solution. 200 grams of perfume oil is added to the previous mixture under mechanical agitation and the temperature is raised to 45° C. After mixing at higher speed until a stable emulsion is obtained, the second solution and 4 grams of sodium sulfate salt are added to the emulsion. This second solution contains 3 grams of polyacrylic acid polymer (Colloid C121, 25% solids (Kemira Chemicals, Inc. Kennesaw, Ga. U.S.A.), 100 grams of distilled water, sodium hydroxide solution to adjust pH to 6.0, 10 grams of partially methylated methyol melamine resin (Cymel 385, 80% Cytec). This mixture is heated till 85C and maintained 8 hours with continuous stirring to complete the encapsulation process. 23 grams of acetoacetamide (Sigma-Aldrich, Saint Louis, Mo. U.S.A.) is added to the suspension. Salts and structuring agents can then still be added to the slurry.

### Example 4

### Process of Making a Polymer Assisted Delivery (PAD) Matrix System

A mixture comprising 50% of a perfume composition comprising one or more Table 1 PRMs, 40% of carboxyl-terminated Hypro™ RLP 1300X18 (CAS#0068891-50-9) from nanoresins, (put at 60° C. in warm water bath for 1 hour before mixing) and 10% of Lupasol® WF(CAS#09002-98-6) from BASF (put at 60° C. in warm water bath for 1 hour before mixing). Mixing is achieved by mixing for five minutes using a Ultra-Turrax T25 Basic equipment (from IKA). After mixing, the mixture is put in a warm water bath at 60° C. for ±12 hours. A homogenous, viscous and sticky material is obtained.

In the same way as described above different ratios between the components can be used:

Weight %

| | | | | | |
|---|---|---|---|---|---|
| Perfume composition | 40 | 50 | 60 | 70 | 80 |
| Lupasol ® WF | 12 | 10 | 8 | 6 | 4 |
| Hypro ™ RLP | 48 | 40 | 32 | 24 | 16 |
| CTBN1300X18 | | | | | |

Weight %

| Perfume composition | 50 | 50 | 50 | 50 | 50 | 50 | 50 | 50 |
|---|---|---|---|---|---|---|---|---|
| Lupasol ® WF | 2.5 | 5 | 7.5 | 10 | 12.5 | 15 | 17.5 | 20 |
| Hypro ™ RLP | 47.5 | 45 | 42.5 | 40 | 37.5 | 35 | 32.5 | 30 |
| 1300X18 | | | | | | | | |

### Examples 5-35

### Product Formulation

Non-limiting examples of product formulations containing PRMs disclosed in the present specification perfume and amines summarized in the following table.

### Examples 5-10

Granular laundry detergent compositions for hand washing or washing machines, typically top-loading washing machines.

| | 5 | 6 | 7 | 8 | 9 | 10 |
|---|---|---|---|---|---|---|
| | (wt %) | (wt %) | (wt %) | (wt %) | (wt %) | (wt %) |
| Linear alkylbenzenesulfonate | 20 | 22 | 20 | 15 | 19.5 | 20 |
| C₁₂₋₁₄ Dimethylhydroxyethyl | 0.7 | 0.2 | 1 | 0.6 | 0.0 | 0 |
| ammonium chloride | | | | | | |
| AE3S | 0.9 | 1 | 0.9 | 0.0 | 0.4 | 0.9 |
| AE7 | 0.0 | 0.0 | 0.0 | 1 | 0.1 | 3 |
| Sodium tripolyphosphate | 5 | 0.0 | 4 | 9 | 2 | 0.0 |
| Zeolite A | 0.0 | 1 | 0.0 | 1 | 4 | 1 |
| 1.6R Silicate (SiO₂:Na₂O at | 7 | 5 | 2 | 3 | 3 | 5 |
| ratio 1.6:1) | | | | | | |
| Sodium carbonate | 25 | 20 | 25 | 17 | 18 | 19 |
| Polyacrylate MW 4500 | 1 | 0.6 | 1 | 1 | 1.5 | 1 |
| Random graft copolymer¹ | 0.1 | 0.2 | 0.0 | 0.0 | 0.05 | 0.0 |
| Carboxymethyl cellulose | 1 | 0.3 | 1 | 1 | 1 | 1 |
| Stainzyme ® (20 mg active/g) | 0.1 | 0.2 | 0.1 | 0.2 | 0.1 | 0.1 |
| Protease (Savinase ®, 32.89 mg | 0.1 | 0.1 | 0.1 | 0.1 | | 0.1 |
| active/g) | | | | | | |
| Amylase - Natalase ® (8.65 mg | 0.1 | 0.0 | 0.1 | 0.0 | 0.1 | 0.1 |
| active/g) | | | | | | |
| Lipase - Lipex ® (18 mg active/g) | 0.03 | 0.07 | 0.3 | 0.1 | 0.07 | 0.4 |
| Fluorescent Brightener 1 | 0.06 | 0.0 | 0.06 | 0.18 | 0.06 | 0.06 |
| Fluorescent Brightener 2 | 0.1 | 0.06 | 0.1 | 0.0 | 0.1 | 0.1 |
| DTPA | 0.6 | 0.8 | 0.6 | 0.25 | 0.6 | 0.6 |
| MgSO₄ | 1 | 1 | 1 | 0.5 | 1 | 1 |
| Sodium Percarbonate | 0.0 | 5.2 | 0.1 | 0.0 | 0.0 | 0.0 |
| Sodium Perborate | 4.4 | 0.0 | 3.85 | 2.09 | 0.78 | 3.63 |
| Monohydrate | | | | | | |
| NOBS | 1.9 | 0.0 | 1.66 | 0.0 | 0.33 | 0.75 |
| TAED | 0.58 | 1.2 | 0.51 | 0.0 | 0.015 | 0.28 |
| Sulphonated zinc | 0.0030 | 0.0 | 0.0012 | 0.0030 | 0.0021 | 0.0 |
| phthalocyanine | | | | | | |
| S-ACMC | 0.1 | 0.0 | 0.0 | 0.0 | 0.06 | 0.0 |
| Direct Violet Dye (DV9 | 0.0 | 0.0 | 0.0003 | 0.0001 | 0.0001 | 0.0 |
| or DV99 or DV66) | | | | | | |
| Additional Neat | 0.5 | 0.5 | 0.5 | 0.5 | 0.5 | 0.5 |
| Perfume ⁽²⁾ | | | | | | |
| Amine ⁽¹⁾ | 0.1 | 0.5 | 0.0 | 0.01 | 0.02 | 0.00 |
| Perfume Delivery System As | 0.05 | 0.0 | 0.1 | 0.0 | 0.2 | 0.4 |
| Disclosed In The Present | | | | | | |
| Specification Including | | | | | | |
| Examples 2-3 | | | | | | |
| Perfume comprising one or | 0.3 | 0.4 | 0.01 | 0.02 | 0.04 | 0.1 |
| more PRMs from Table 1 | | | | | | |

| | |
|---|---|
| Sulfate/Moisture | Balance |

⁽¹⁾ One or more materials comprising an amine moiety as disclosed in the present specification.
⁽²⁾ Optional.

### Examples 11-16

Granular laundry detergent compositions typically for front-loading automatic washing machines.

| | 11 | 12 | 13 | 14 | 15 | 16 |
|---|---|---|---|---|---|---|
| | (wt %) | (wt %) | (wt %) | (wt %) | (wt %) | (wt %) |
| Linear alkylbenzenesulfonate | 8 | 7.1 | 7 | 6.5 | 7.5 | 7.5 |
| AE3S | 0 | 4.8 | 1.0 | 5.2 | 4 | 4 |
| C₁₂₋₁₄ Alkylsulfate | 1 | 0 | 1 | 0 | 0 | 0 |
| AE7 | 2.2 | 0 | 2.2 | 0 | 0 | 0 |
| C₁₀₋₁₂ Dimethyl | 0.75 | 0.94 | 0.98 | 0.98 | 0 | 0 |
| hydroxyethylammonium chloride | | | | | | |
| Crystalline layered silicate (δ- | 4.1 | 0 | 4.8 | 0 | 0 | 0 |
| Na₂Si₂O₅) | | | | | | |
| Zeolite A | 5 | 0 | 5 | 0 | 2 | 2 |
| Citric Acid | 3 | 5 | 3 | 4 | 2.5 | 3 |
| Sodium Carbonate | 15 | 20 | 14 | 20 | 23 | 23 |
| Silicate 2R (SiO₂:Na₂O at ratio 2:1) | 0.08 | 0 | 0.11 | 0 | 0 | 0 |
| Soil release agent | 0.75 | 0.72 | 0.71 | 0.72 | 0 | 0 |
| Acrylic Acid/Maleic Acid Copolymer | 1.1 | 3.7 | 1.0 | 3.7 | 2.6 | 3.8 |
| Carboxymethylcellulose | 0.15 | 1.4 | 0.2 | 1.4 | 1 | 0.5 |
| Protease - Purafect ® (84 mg active/g) | 0.2 | 0.2 | 0.3 | 0.15 | 0.12 | 0.13 |
| Amylase - Stainzyme Plus ® (20 mg | 0.2 | 0.15 | 0.2 | 0.3 | 0.15 | 0.15 |
| active/g) | | | | | | |
| Lipase - Lipex ® (18.00 mg active/g) | 0.05 | 0.15 | 0.1 | 0 | 0 | 0 |
| Amylase - Natalase ® (8.65 mg | 0.1 | 0.2 | 0 | 0 | 0.15 | 0.15 |
| active/g) | | | | | | |
| Cellulase - Celluclean ™ (15.6 mg | 0 | 0 | 0 | 0 | 0.1 | 0.1 |
| active/g) | | | | | | |
| TAED | 3.6 | 4.0 | 3.6 | 4.0 | 2.2 | 1.4 |
| Percarbonate | 13 | 13.2 | 13 | 13.2 | 16 | 14 |
| Na salt of Ethylenediamine-N,N′- | 0.2 | 0.2 | 0.2 | 0.2 | 0.2 | 0.2 |
| disuccinic acid, (S,S) isomer (EDDS) | | | | | | |
| Hydroxyethane di phosphonate (HEDP) | 0.2 | 0.2 | 0.2 | 0.2 | 0.2 | 0.2 |
| MgSO₄ | 0.42 | 0.42 | 0.42 | 0.42 | 0.4 | 0.4 |
| Perfume | 0.5 | 0.6 | 0.5 | 0.6 | 0.6 | 0.6 |
| Suds suppressor agglomerate | 0.05 | 0.1 | 0.05 | 0.1 | 0.06 | 0.05 |
| Soap | 0.45 | 0.45 | 0.45 | 0.45 | 0 | 0 |
| Sulphonated zinc phthalocyanine | 0.0007 | 0.0012 | 0.0007 | 0 | 0 | 0 |
| (active) | | | | | | |
| S-ACMC | 0.01 | 0.01 | 0 | 0.01 | 0 | 0 |
| Direct Violet 9 (active) | 0 | 0 | 0.0001 | 0.0001 | 0 | 0 |
| Additional Neat Perfume ⁽²⁾ | 0.5 | 0.5 | 0.5 | 0.5 | 0.5 | 0.5 |
| Amine ⁽¹⁾ | 0.1 | 0.5 | 0.0 | 0.01 | 0.02 | 0.00 |
| Perfume Delivery System As Disclosed | 0.05 | 0.0 | 0.1 | 0.0 | 0.2 | 0.4 |
| In The Present Specification Including | | | | | | |
| Examples 2-3 | | | | | | |
| Perfume comprising one or more PRMs | 0.3 | 0.4 | 0.01 | 0.02 | 0.04 | 0.1 |
| from Table 1 | | | | | | |

| | |
|---|---|
| Sulfate/Water & Miscellaneous | Balance |

⁽¹⁾ One or more materials comprising an amine moiety as disclosed in the present specification.
⁽²⁾ Optional.

The typical pH is about 10.

### Examples 17-23

### Heavy Duty Liquid Laundry Detergent Compositions

| | 17 | 18 | 19 | 20 | 21 | 22 | 23 |
|---|---|---|---|---|---|---|---|
| | (wt %) | (wt %) | (wt %) | (wt %) | (wt %) | (wt %) | (wt %) |
| AES C₁₂₋₁₅ alkyl ethoxy (1.8) | 11 | 10 | 4 | 6.32 | 0 | 0 | 0 |
| sulfate | | | | | | | |
| AE3S | 0 | 0 | 0 | 0 | 2.4 | 0 | 0 |
| Linear alkyl benzene | 1.4 | 4 | 8 | 3.3 | 5 | 8 | 19 |
| sulfonate/sulfonic acid | | | | | | | |
| HSAS | 3 | 5.1 | 3 | 0 | 0 | 0 | 0 |
| Sodium formate | 1.6 | 0.09 | 1.2 | 0.04 | 1.6 | 1.2 | 0.2 |
| Sodium hydroxide | 2.3 | 3.8 | 1.7 | 1.9 | 1.7 | 2.5 | 2.3 |
| Monoethanolamine | 1.4 | 1.49 | 1.0 | 0.7 | 0 | 0 | To pH 8.2 |
| Diethylene glycol | 5.5 | 0.0 | 4.1 | 0.0 | 0 | 0 | 0 |
| AE9 | 0.4 | 0.6 | 0.3 | 0.3 | 0 | 0 | 0 |
| AE8 | 0 | 0 | 0 | 0 | 0 | 0 | 20.0 |
| AE7 | 0 | 0 | 0 | 0 | 2.4 | 6 | 0 |
| Chelant (HEDP) | 0.15 | 0.15 | 0.11 | 0.07 | 0.5 | 0.11 | 0.8 |
| Citric Acid | 2.5 | 3.96 | 1.88 | 1.98 | 0.9 | 2.5 | 0.6 |
| C₁₂₋₁₄ dimethyl Amine Oxide | 0.3 | 0.73 | 0.23 | 0.37 | 0 | 0 | 0 |
| C₁₂₋₁₈ Fatty Acid | 0.8 | 1.9 | 0.6 | 0.99 | 1.2 | 0 | 15.0 |
| 4-formyl-phenylboronic acid | 0 | 0 | 0 | 0 | 0.05 | 0.02 | 0.01 |
| Borax | 1.43 | 1.5 | 1.1 | 0.75 | 0 | 1.07 | 0 |
| Ethanol | 1.54 | 1.77 | 1.15 | 0.89 | 0 | 3 | 7 |
| A compound having the following | 0.1 | 0 | 0 | 0 | 0 | 0 | 2.0 |
| general structure: | | | | | | | |
| bis((C₂H₅O)(C₂H₄O)n)(CH₃)—N⁺—CₓH₂ₓ—N⁺—(CH₃)— | | | | | | | |
| bis((C₂H₅O)(C₂H₄O)n), wherein n = | | | | | | | |
| from 20 to 30, and x = from 3 to | | | | | | | |
| 8, or sulphated or sulphonated | | | | | | | |
| variants thereof | | | | | | | |
| Ethoxylated (EO₁₅) tetraethylene | 0.3 | 0.33 | 0.23 | 0.17 | 0.0 | 0.0 | 0 |
| pentamine | | | | | | | |
| Ethoxylated Polyethylenimine | 0 | 0 | 0 | 0 | 0 | 0 | 0.8 |
| Ethoxylated hexamethylene | 0.8 | 0.81 | 0.6 | 0.4 | 1 | 1 | |
| diamine | | | | | | | |
| 1,2-Propanediol | 0.0 | 6.6 | 0.0 | 3.3 | 0.5 | 2 | 8.0 |
| Fluorescent Brightener | 0.2 | 0.1 | 0.05 | 0.3 | 0.15 | 0.3 | 0.2 |
| Hydrogenated castor oil derivative | 0.1 | 0 | 0 | 0 | 0 | 0 | 0.1 |
| structurant | | | | | | | |
| Perfume | 1.6 | 1.1 | 1.0 | 0.8 | 0.9 | 1.5 | 1.6 |
| Protease (40.6 mg active/g) | 0.8 | 0.6 | 0.7 | 0.9 | 0.7 | 0.6 | 1.5 |
| Mannanase: Mannaway ® (25 mg | 0.07 | 0.05 | 0.045 | 0.06 | 0.04 | 0.045 | 0.1 |
| active/g) | | | | | | | |
| Amylase: Stainzyme ® (15 mg | 0.3 | 0 | 0.3 | 0.1 | 0 | 0.4 | 0.1 |
| active/g) | | | | | | | |
| Amylase: Natalase ® (29 mg | 0 | 0.2 | 0.1 | 0.15 | 0.07 | 0 | 0.1 |
| active/g) | | | | | | | |
| Xyloglucanase (Whitezyme ®, | 0.2 | 0.1 | 0 | 0 | 0.05 | 0.05 | 0.2 |
| 20 mg active/g) | | | | | | | |
| Lipex ® (18 mg active/g) | 0.4 | 0.2 | 0.3 | 0.1 | 0.2 | 0 | 0 |
| Additional Neat Perfume ⁽²⁾ | 0.5 | 0.5 | 0.5 | 0.5 | 0.5 | 0.5 | 0.5 |
| Amine ⁽¹⁾ | 0.1 | 0.5 | 0.0 | 0.01 | 0.02 | 0.00 | 0.07 |
| Perfume Delivery System As | 0.05 | 0.0 | 0.1 | 0.0 | 0.2 | 0.4 | 0.0 |
| Disclosed In The Present | | | | | | | |
| Specification Including Examples | | | | | | | |
| 2-3 | | | | | | | |
| Perfume comprising one or more | 0.7 | 0.5 | 0.8 | 0.05 | 0.6 | 0.1 | 0.6 |
| PRMs from Table 1 | | | | | | | |

| | |
|---|---|
| *Water, dyes & minors | Balance |

*Based on total cleaning and/or treatment composition weight, a total of no more than 12% water
⁽¹⁾ One or more materials comprising an amine moiety as disclosed in the present specification.
⁽²⁾ Optional.

### Examples 24-25

### Unit Dose Compositions

| | 24 | 25 |
|---|---|---|
| C₁₄-C₁₅ alkyl poly ethoxylate (8) | 12 | — |
| C₁₂-C₁₄ alkyl poly ethoxylate (7) | 1 | 14 |
| C₁₂-C₁₄ alkyl poly ethoxylate (3) sulfate Mono | 8.4 | 9 |
| EthanolAmine salt | | |
| Linear Alkylbenzene sulfonic acid | 15 | 16 |
| Citric Acid | 0.6 | 0.5 |
| C₁₂-C₁₈ Fatty Acid | 15 | 17 |
| Enzymes | 1.5 | 1.2 |
| PEI 600 EO20 | 4 | — |
| Diethylene triamine penta methylene | 1.3 | — |
| phosphonic acid or HEDP | | |
| Fluorescent brightener | 0.2 | 0.3 |
| Hydrogenated Castor Oil | 0.2 | 0.2 |
| 1,2 propanediol | 16 | 12 |
| Glycerol | 6.2 | 8.5 |
| Sodium hydroxide | — | 1 |
| Mono Ethanol Amine | 7.9 | 6.1 |
| Dye | Present | Present |
| PDMS | — | 2.7 |
| Potassium sulphite | 0.2 | 0.2 |
| Additional Neat Perfume⁽²⁾ | 0.5 | 0.5 |
| Amine⁽¹⁾ | 0.1 | 0.5 |
| Perfume Delivery System As Disclosed In The | 0.05 | 0.0 |
| Present Specification Including Examples 2-3 | | |
| Perfume comprising one or more PRMs from | 0.3 | 0.4 |
| Table 1 | | |
| Water | Up to | Up to |
| | 100p | 100 |

⁽¹⁾One or more materials comprising an amine moiety as disclosed in the present specification.
⁽²⁾Optional.

### Example 26-31

### Bleach & Laundry Additive Detergent Formulations

| | 26 | 27 | 28 | 29 | 30 | 31 |
|---|---|---|---|---|---|---|
| AES | 11.3 | 6.0 | 15.4 | 16.0 | 12.0 | 10.0 |
| LAS | 25.6 | 12.0 | 4.6 | — | — | 26.1 |
| MEA-HSAS | — | — | — | 3.5 | — | — |
| DTPA: Diethylene | 0.51 | — | 1.5 | — | — | 2.6 |
| triamine pentaacetic | | | | | | |
| acid | | | | | | |
| 4,5-Dihydroxy-1,3- | 1.82 | — | — | — | — | 1.4 |
| benzenedisulfonic | | | | | | |
| acid disodium salt | | | | | | |
| 1,2-propandiol | — | 10 | — | — | — | 15 |
| Copolymer of | 2.0 | | | | | |
| dimethylterephthalate, | | | | | | |
| 1,2-propylene | | | | | | |
| glycol, methyl | | | | | | |
| capped PEG | | | | | | |
| Poly(ethyleneimine) | | 1.8 | | | | |
| ethoxylated, PEI600 | | | | | | |
| E20 | | | | | | |
| Acrylic acid/maleic | | | 2.9 | | | |
| acid copolymer | | | | | | |
| Acusol 880 | | | | 2.0 | 1.8 | 2.9 |
| (Hydrophobically | | | | | | |
| Modified Non-Ionic | | | | | | |
| Polyol) | | | | | | |
| Protease (55 mg/g | — | — | — | — | 0.1 | 0.1 |
| active) | | | | | | |
| Amylase (30 mg/g | — | — | — | — | — | 0.02 |
| active) | | | | | | |
| Brightener | 0.21 | — | — | 0.15 | — | 0.18 |
| Dye or mixture or | 0.01 | 0.005 | 0.006 | 0.002 | 0.007 | 0.008 |
| dyes selected from | | | | | | |
| Examples 1-28 in | | | | | | |
| Table 1. | | | | | | |
| Additional Neat | 0.5 | 0.5 | 0.5 | 0.5 | 0.5 | 0.5 |
| Perfume ⁽²⁾ | | | | | | |
| Amine ⁽¹⁾ | 0.1 | 0.5 | 0.0 | 0.01 | 0.02 | 0.00 |
| Perfume Delivery | 0.05 | 0.0 | 0.1 | 0.0 | 0.2 | 0.4 |
| System As Disclosed | | | | | | |
| In The Present | | | | | | |
| Specification | | | | | | |
| Including Examples | | | | | | |
| 2-3 | | | | | | |
| Perfume comprising | 0.3 | 0.4 | 0.01 | 0.02 | 0.04 | 0.1 |
| one or more PRMs | | | | | | |
| from Table 1 | | | | | | |
| water, other optional | to 100% | to 100% | to 100% | to 100% | to 100% | to 100% |
| agents/components* | balance | balance | balance | balance | balance | balance |

⁽³⁾ One or more materials comprising an amine moiety as disclosed in the present specification.
⁽⁴⁾ Optional.
*Other optional agents/components include suds suppressors, structuring agents such as those based on Hydrogenated Castor Oil (preferably Hydrogenated Castor Oil, Anionic Premix), solvents and/or Mica pearlescent aesthetic enhancer.

Raw Materials and Notes For Composition Examples
LAS is linear alkylbenzenesulfonate having an average aliphatic carbon chain length C₉-C₁₅ supplied by Stepan, Northfield, Ill., USA or Huntsman Corp. (HLAS is acid form).
C₁₂₋₁₄ Dimethylhydroxyethyl ammonium chloride, supplied by Clariant GmbH, Germany
AE3S is C₁₂₋₁₅ alkyl ethoxy (3) sulfate supplied by Stepan, Northfield, Ill., USA
AE7 is C₁₂₋₁₅ alcohol ethoxylate, with an average degree of ethoxylation of 7, supplied by Huntsman, Salt Lake City, Utah, USA
AES is C-₁₀₋₁₈ alkyl ethoxy sulfate supplied by Shell Chemicals.
AE9 is C₁₂₋₁₃ alcohol ethoxylate, with an average degree of ethoxylation of 9, supplied by Huntsman, Salt Lake City, Utah, USA
HSAS or HC1617HSAS is a mid-branched primary alkyl sulfate with average carbon chain length of about 16-17
Sodium tripolyphosphate is supplied by Rhodia, Paris, France
Zeolite A is supplied by Industrial Zeolite (UK) Ltd, Grays, Essex, UK
1.6R Silicate is supplied by Koma, Nestemica, Czech Republic
Sodium Carbonate is supplied by Solvay, Houston, Tex., USA
Polyacrylate MW 4500 is supplied by BASF, Ludwigshafen, Germany
Carboxymethyl cellulose is Finnfix® V supplied by CP Kelco, Arnhem, Netherlands
Suitable chelants are, for example, diethylenetetraamine pentaacetic acid (DTPA) supplied by Dow Chemical, Midland, Mich., USA or Hydroxyethane diphosphonate (HEDP) supplied by Solutia, St Louis, Mo., USA Bagsvaerd, Denmark
Savinase®, Natalase®, Stainzyme®, Lipex®, Celluclean™, Mannaway® and Whitezyme® are all products of Novozymes, Bagsvaerd, Denmark.
Proteases may be supplied by Genencor International, Palo Alto, Calif., USA (e.g. Perfect Prime®) or by Novozymes, Bagsvaerd, Denmark (e.g. Liquanase®, Coronase®).
Fluorescent Brightener 1 is Tinopal® AMS, Fluorescent Brightener 2 is Tinopal® CBS-X, Sulphonated zinc phthalocyanine and Direct Violet 9 is Pergasol® Violet BN-Z all supplied by Ciba Specialty Chemicals, Basel, Switzerland
Sodium percarbonate supplied by Solvay, Houston, Tex., USA
Sodium perborate is supplied by Degussa, Hanau, Germany
NOBS is sodium nonanoyloxybenzenesulfonate, supplied by Future Fuels, Batesville, USA
TAED is tetraacetylethylenediamine, supplied under the Peractive® brand name by Clariant GmbH, Sulzbach, Germany
S-ACMC is carboxymethylcellulose conjugated with C.I. Reactive Blue 19, sold by Megazyme, Wicklow, Ireland under the product name AZO-CM-CELLULOSE, product code S-ACMC.
Soil release agent is Repel-o-tex® PF, supplied by Rhodia, Paris, France
Acrylic Acid/Maleic Acid Copolymer is molecular weight 70,000 and acrylate:maleate ratio 70:30, supplied by BASF, Ludwigshafen, Germany
Na salt of Ethylenediamine-N,N′-disuccinic acid, (S,S) isomer (EDDS) is supplied by Octel, Ellesmere Port, UK
Hydroxyethane di phosphonate (HEDP) is supplied by Dow Chemical, Midland, Mich., USA
Suds suppressor agglomerate is supplied by Dow Corning, Midland, Mich., USA
HSAS is mid-branched alkyl sulfate as disclosed in U.S. Pat. No. 6,020,303 and U.S. Pat. No. 6,060,443
C₁₂₋₁₄ dimethyl Amine Oxide is supplied by Procter & Gamble Chemicals, Cincinnati, USA
Random graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40:60 and no more than 1 grafting point per 50 ethylene oxide units.
Ethoxylated polyethyleneimine is polyethyleneimine (MW=600) with 20 ethoxylate groups per —NH.
Cationic cellulose polymer is LK400, LR400 and/or JR30M from Amerchol Corporation, Edgewater N.J.
Note: all enzyme levels are expressed as % enzyme raw material

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

### Example 32

### Shampoo Formulations

| Ingredient | |
|---|---|
| Ammonium Laureth Sulfate (AE3S) | 6.00 |
| Ammonium Lauryl Sulfate (ALS) | 10.00 |
| Laureth-4 Alcohol | 0.90 |
| Trihydroxystearin⁽⁷⁾ | 0.10 |
| Perfume comprising one or more | 0.60 |
| PRMs from Table 1 | |
| Sodium Chloride | 0.40 |
| Citric Acid | 0.04 |
| Sodium Citrate | 0.40 |
| Sodium Benzoate | 0.25 |
| Ethylene Diamine Tetra Acetic Acid | 0.10 |
| Dimethicone^{(9,10,11)} | 1.00⁽⁹⁾ |
| Water and Minors (QS to 100%) | Balance |

### Example 33-35

### Fine Fragrance Formulations

| Ingredient | 33 | 34 | 35 |
|---|---|---|---|
| Cyclic oligosaccharide | 0 | 5 | 10 |
| Ethanol | 90 | 75 | 80 |
| Perfume comprising one or more | 10 | 20 | 10 |
| PRMs from Table 1 | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A perfume delivery system comprising from 0.001% to about 50%, of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof; wherein said perfume delivery system is selected from a polymer assisted delivery system; a molecule-assisted delivery system; a fiber-assisted delivery system; an amine assisted delivery system; a cyclodextrin delivery system; a starch encapsulated accord; an inorganic carrier delivery system; or a pro-perfume.

2. The perfume delivery system of claim 1 comprising from 0.01% to about 10% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

3. The perfume delivery system of claim 1 comprising from 0.025% to about 1% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

4. A perfume delivery system according to claim 1, said perfume delivery system being a nanocapsule or a microcapsule comprising, based on total nanocapsule or microcapsule weight, from about 0.1% to about 99% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

5. A perfume delivery system according to claim 4, said perfume delivery system being a nanocapsule or a microcapsule comprising, based on total nanocapsule or microcapsule weight, from 30 to about 90% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

6. A perfume delivery system according to claim 4, said perfume delivery system being a nanocapsule or a microcapsule comprising, based on total nanocapsule or microcapsule weight, from 65% to about 90% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

7. A perfume delivery system according to claim 1, said perfume delivery system being a starch encapsulated accord comprising, based on total starch encapsulate or starch agglomerate weight, from about 0.1% to about 99% of one or more molecules selected from the group consisting of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

8. A perfume delivery system according to claim 7, said perfume delivery system being a starch encapsulated accord comprising, based on total starch encapsulate or starch agglomerate weight, from 30 to about 90% of one or more molecules selected from the group consisting of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

9. A perfume delivery system according to claim 7, said perfume delivery system being a starch encapsulated accord comprising, based on total starch encapsulate or starch agglomerate weight from 65% to about 90% of one or more molecules selected from the group consisting of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

10. A perfume delivery system according to claim 1, said perfume delivery system being a cyclodextrin delivery system comprising based on total cyclodextrin delivery system weight, from 0.1% to about 99% of one or more molecules selected from the group consisting ofone or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

11. A perfume delivery system according to claim 10, said perfume delivery system being a cyclodextrin delivery system comprising based on total cyclodextrin delivery system weight from 5% to about 60% of one or more molecules selected from the group consisting ofone or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

12. A perfume delivery system according to claim 10, said perfume delivery system being a cyclodextrin delivery system comprising based on total cyclodextrin delivery system weight from 5% to about 25% of one or more molecules selected from the group consisting ofone or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

13. A perfume delivery system according to claim 1, said perfume delivery system being a polymer assisted delivery matrix system comprising, based on total polymer assisted delivery matrix system weight, from 0.1% to about 99% of one or more molecules selected from the group consisting of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

14. A perfume delivery system according to claim 13, said perfume delivery system being a polymer assisted delivery matrix system comprising, based on total polymer assisted delivery matrix system weight from 5% to about 60% of one or more molecules selected from the group consisting of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

15. A perfume delivery system according to claim 13, said perfume delivery system being a polymer assisted delivery matrix system comprising, based on total polymer assisted delivery matrix system weight from 5% to about 25% of one or more molecules selected from the group consisting of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

16. A perfume delivery system according to claim 1, said perfume delivery system being an amine assisted delivery system, said amine assisted delivery system comprising, based on total amine assisted delivery system weight, from 1% to about 99% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

17. A perfume delivery system according to claim 16, said perfume delivery system being an amine assisted delivery system, said amine assisted delivery system comprising, based on total amine assisted delivery system weight from 5% to about 60% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

18. A perfume delivery system according to claim 16, said perfume delivery system being an amine assisted delivery system, said amine assisted delivery system comprising, based on total amine assisted delivery system weight from 5% to about 25% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

19. A perfume delivery system according to claim 1, said perfume delivery system being a pro-perfume amine reaction product, said pro-perfume amine reaction product comprising, based on total pro-perfume amine reaction product weight, from 0.1% to about 99% of one or more molecules selected from the group consisting of Furaneol carbonate;
stereoisomers and Furaneol carbonate; and mixtures thereof.

20. A perfume delivery system according to claim 19, said perfume delivery system being an amine assisted delivery system, said amine assisted delivery system comprising, based on total amine assisted delivery system weight from 5% to about 60% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

21. A perfume delivery system according to claim 19, said perfume delivery system being an amine assisted delivery system, said amine assisted delivery system comprising, based on total amine assisted delivery system weight, from 5% to about 25% of one or more molecules selected from the group consisting of 2-benzyl, 4-isopropyl, 5-dimethyl, m-dioxane and Furaneol carbonate; stereoisomers of 2-benzyl, 4-isopropyl, 5-dimethyl, m-dioxane and Furaneol carbonate; and mixtures thereof.

22. A consumer product comprising, based on total consumer product weight, from about 0.001% to about 20% of a perfume delivery system selected from the perfume delivery systems of claims 1-21 and mixtures thereof.

23. A consumer product according to claim 22, comprising, based on total consumer product weight from about 0.1% to about 0.5% of a perfume delivery system selected from the perfume delivery systems of claims 1-21 and mixtures thereof.
